(19) United States
(12) Patent Application Publication
(10) Pub. No.: US 2005/0159762 A1
(43) Pub. Date: Jul. 21, 2005

(54) Suture arrow device and installation device

(76) Inventors: Juha-Pekka Nuutinen, Tampere (FI); Peter C. Miller, Largo, FL (US); Hugh West, Salt Lake City, UT (US); Walter Shelton, Jackson, MS (US)

Correspondence Address: KENYON & KENYON, 1 BROADWAY, NEW YORK, NY, 10004, US

(21) Appl. No.: 10/979,166

(22) Filed: Nov. 3, 2004

Related U.S. Application Data
(60) Provisional application No. 60/534,094, filed on Dec. 30, 2003.

(51) Int. Cl. A61B 17/04; A61D 1/00; A61B 17/08

(52) U.S. Cl. 606/148; 606/228; 606/232

(57) ABSTRACT

A suture arrow device is provided that includes a first and second arrow connecting for a flexible connecting member. A method of using the suture arrow device to repair damaged tissue is also provided. An installation device for inserting two suture arrow sequentially and a method of using such an installation device to repair tissue is also provided.

### CROSS-REFERENCE TO RELATED APPLICATIONS

This application claims priority to U.S. Provisional Application No. 60/534,094, filed on Dec. 30, 2003.

### FIELD OF THE INVENTION

The present invention relates to a suture arrow device and an installation device for sequentially installing suture arrows.

### BACKGROUND

It has been shown that fixation of meniscus traumas, like ruptures and lesions by suturing gives better results than the removal of traumatized meniscal tissue. However, arthroscopic suturing is a complicated and tedious technique where risks for the patient are significant because of danger of damaging vessels and nerves. Therefore, for a long time, surgeons have desired a meniscus lesion fixation device which may be used more rapidly and safely than sutures. Although meniscal lesion fixation devices are known, such devices suffer from disadvantages because they have, for example, configurations pr components that require complicated surgical techniques or configurations or components that can potentially damage the tissue. In particular, the base and anchoring or implantation members of these devices are inserted side-by-side and simultaneously, making such devices inflexible and creating the risk of operational trauma. Some of these devices require a difficult arthroscopic knot tying, clipping or locking procedure which leaves tied knots, clips, or locking mechanisms on the surface or near the surface of the tissue, which can damage the tissue.

Accordingly, there is a need for fixation device that allows a minimally invasive method for repairing a tissue and that is rapid, easy to install, and provides strong and safe fixation of a tissue tear. There is also a need for an installation device for precisely and safely inserting a fixation device.

### SUMMARY OF THE INVENTION

In an embodiment, the present invention provides a suture arrow device comprising a first tissue arrow, a second tissue arrow, and a flexible connecting member connecting the first and second tissue arrows. The first tissue arrow comprise a single, elongated body shaped like an arrow and having a pointed end and the second tissue arrow comprising a single, elongated body shaped like an arrow and having at least one protrusion thereon and a pointed end.

In another embodiment, the present invention provides a method of repairing a body tissue rupture comprising providing a suture arrow device comprising a first tissue arrow, a second tissue arrow having at least one protrusion thereon, and a flexible connecting member connecting the first and second tissue arrows. The method further comprises inserting the first tissue arrow through the body tissue and seating the first tissue arrow on the outer surface of the body tissue such that the first tissue arrow is substantially perpendicular to the flexible connecting member. The method further comprises inserting the second tissue arrow into the body tissue in a location off-set from the first tissue arrow thereby closing the rupture.

In another embodiment, the present invention provides an installation device comprising an elongated cannulated slide piece having a proximal portion and a distal portion terminating in a sharp tip. The proximal portion has a lateral surface defining a longitudinally extending groove. The groove has sequential gradations and a notch proximal of the sequential gradations. The installation device further comprises a spring located about the proximal portion of the slide piece, a pusher rod housed within the slide piece, and a casing disposed about the proximal portion of the slide piece. A button is housed by the casing and is assessable to the longitudinally extending groove of the lateral surface of the proximal portion of the slide piece.

In another embodiment, the present invention provides an installation device comprising a slide assembly comprising an elongated cannulated inner slide piece and an outer slide piece. The inner slide piece has a distal portion terminating in a sharp tip and a proximal portion having a lateral surface defining sequential gradations and a notch proximal of the sequential gradations. The outer slide piece is disposed about the proximal portion of the inner slide piece and has a side surface including a flange and defining a longitudinally extending slit that exposes the lateral surface of the proximal portion of the inner slide piece. The installation device further comprises an outer spring located about the outer slide piece and an inner spring at a proximal end of the inner slide piece. The installation device further comprises a tab member located about the inner slide piece, a pusher rod housed within the inner slide piece, and a casing disposed about the outer slide piece. A button is housed by the casing and is assessable to the lateral surface of the proximal portion of the inner slide piece and the side surface of the outer slide piece.

### BRIEF DESCRIPTION OF THE DRAWINGS

FIG. 1a is a side view of an embodiment of a suture arrow device of the present invention indicating dimensions of the suture arrow device.

FIG. 2 depicts a step of a method of installing a suture arrow device of the present invention into damaged tissue.

FIG. 3 depicts a step subsequent to the step depicted in FIG. 2.

FIG. 4 depicts a step subsequent to the step depicted in FIG. 3.

FIG. 5 depicts a step subsequent to the step depicted in FIG. 4.

FIG. 6 depicts a step subsequent to the step depicted in FIG. 5.

FIG. 7 depicts a step subsequent to the step depicted in FIG. 6.

FIG. 8 depicts a step subsequent to the step depicted in FIG. 7.

FIG. 9 depicts a step subsequent to the step depicted in FIG. 8.

FIG. 10 is a perspective view of an embodiment of an installation device of the present invention.

FIG. 11 is a perspective view of another embodiment of an installation device of the present invention.

FIG. 11a is a bottom partial view of an installation device of the present invention.

FIG. 11b is a side partial view of an installation device of the present invention.

FIG. 12 is a top view of a proximal portion of a slide piece and a spring located about the proximal portion of the slide piece of an embodiment of an installation device of the present invention.

FIG. 13a is a side view of a pusher rod of an installation device of the present invention.

FIG. 13b is a side view of a pusher rod attached to an end cap of an installation device of the present invention.

FIG. 14 is a side view of a casing of an installation device of the present invention.

FIG. 15 is a front view of a button of an installation device of the present invention.

FIG. 16 is a perspective view of a casing depicting the placement of a button in a housing defined by the casing of an installation device of the present invention.

FIG. 17 is a cross-sectional and partial cut-away view of an embodiment of an installation device of the present invention in a non-operative resting position.

FIG. 18 is a cross-sectional and partial cut-away view of an embodiment of an installation device of the present invention during a step of a method of using the installation device to repair tissue.

FIG. 19 is a cross-sectional and partial cut-away view of an embodiment of an installation device of the present invention during a step subsequent to the step depicted in FIG. 18 of a method of using the installation device to repair tissue.

FIG. 20 is a cross-sectional and partial cut-away view of an embodiment of an installation device of the present invention during a step subsequent to the step depicted in FIG. 19 of a method of using the installation device to repair tissue.

FIG. 21 is a cross-sectional and partial cut-away view of an embodiment of an installation device of the present invention during a step subsequent to the step depicted in FIG. 20 of a method of using the installation device to repair tissue.

FIG. 22 is a side view of an embodiment of an installation device of the present invention.

FIG. 23 is a perspective view of an inner slide piece of an embodiment of an installation device of the present invention.

FIG. 24 is a perspective view of an outer slide piece of an embodiment of an installation device of the present invention.

FIG. 24a is an end view of an outer slide piece of an embodiment of an installation device of the present invention.

FIG. 25 is a perspective view of a tab member of an embodiment of an installation device of the present invention.

FIG. 26 is a top partial view of a slide assembly and an outer spring disposed about an outer slide piece of an embodiment of an installation device of the present invention.

FIG. 27 is a side partial view of a slide assembly, inner spring, and cap of an embodiment of an installation device of the present invention.

FIG. 28 is a perspective view of a cap of an embodiment of an installation device of the present invention.

FIG. 29 is a bottom view of the cap depicted in FIG. 28.

FIG. 30 is a cross-sectional and partial cut-away view of an embodiment of an installation device of the present invention in a non-operative resting position.

FIG. 31 is a cross-sectional and partial cut-away view of an embodiment of an installation device of the present invention during a step of a method of using the installation device to repair tissue.

FIG. 32 is a cross-sectional and partial cut-away view of an embodiment of an installation device of the present invention during a step subsequent to the step depicted in FIG. 31 of a method of using the installation device to repair tissue.

FIG. 33 is a cross-sectional and partial cut-away view of an embodiment of an installation device of the present invention during a step subsequent to the step depicted in FIG. 32 of a method of using the installation device to repair tissue.

FIG. 34 is a cross-sectional and partial cut-away view of an embodiment of an installation device of the present invention during a step subsequent to the step depicted in FIG. 33 of a method of using the installation device to repair tissue.

FIG. 35 is a cross-sectional and partial cut-away view of an embodiment of an installation device of the present invention during a step subsequent to the step depicted in FIG. 34 of a method of using the installation device to repair tissue.

### DETAILED DESCRIPTION OF THE INVENTION

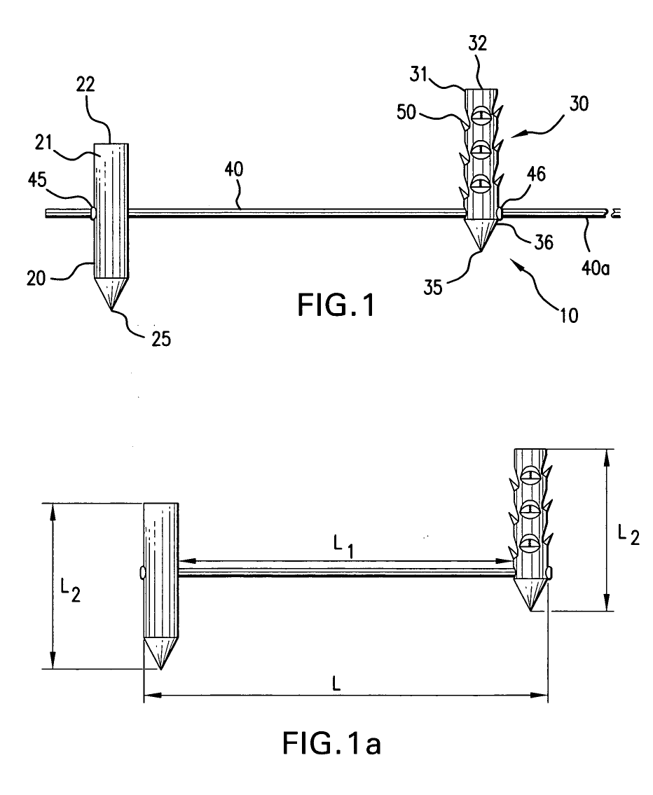

Referring to FIG. 1, an embodiment of the present invention provides a suture arrow device 10 comprising a first tissue arrow 20 and a second tissue arrow 30 and a flexible connecting member 40 connecting first tissue arrow 20 and second tissue arrow 30. First tissue arrow 20 comprises a single, elongated body 21 shaped like an arrow and having a pointed end 25 and second tissue arrow 30 comprises a single, elongated body 31 shaped like an arrow and having at least one protrusion 50, and preferably a plurality of protrusions 50 thereon, and having a pointed end 35. Protrusions 50 may have any suitable shape such as protruding ridges, barbs, pyramids, screw threads, and the like. Although FIG. 1 shows suture arrow device 10 having only two tissues arrows, suture arrow device 10 may have any number of tissue arrows connected by any number of flexible connecting members.

Although tissue arrows 20 and 30 may have any suitable dimensions for tissue repair, preferably the inner diameter of bodies 21 and 31 of first and second tissue arrow 20 and 30 is between about 0.5 millimeters (mm) and 3 mm, and more preferably is 1.5 mm. Referring to FIG. 1a, preferably, the length L₂ of first and second tissue arrows 20 and 30 is between about 5 and 10 mm, and more preferably in 7.5 mm. Preferably, the overall length L of suture arrow device 10 is between about 20 mm and 70 mm.

Referring still to FIG. 1, preferably, a first end 45 of flexible connecting member 40 attaches at a point near the middle (and more preferably at the mid-point) of first tissue arrow 20 and a second end 46 of flexible connecting member 40 attaches at a point near a distal end 36 of second tissue arrow 30 but proximal to pointed end 35 of second tissue arrow 30.

Although flexible connecting member 40 may have any dimensions suitable for tissue repair, preferably the diameter of flexible connecting member is about 0.2 to 3 mm and even more preferably 0.3 to 1 mm. Flexible connecting member 40 may have a fixed length L₁ that is preferably from about 10 to 50 mm and more preferably between about 15 and 25 mm. Even more preferably, flexible connecting member 40 is manufactured in predetermined lengths of 12, 16, or 20 mm (from which the surgeon could then select during the medical procedure in which suture arrow device 10 is employed).

During manufacture of suture arrow device 10, flexible connecting member 40 may be threaded through holes in tissue arrows 20 and 30 and attached to tissue arrows 20 and 30 through the use of knots, with the remainder of flexible connecting member 40 cut away once the desired length of flexible connecting member 40 has been selected (thus, the desired length can be preselected during the manufacturing process and the suture arrow devices provided in various, pre-set lengths for use by a surgeon). Preferably, tissue arrows 20 and 30 have 0.5 mm eyelets for receiving the flexible connecting member.

Suture arrow device 10 may be manufactured of any suitable material known in the art such as a permanent or bioabsorbable material including bioabsorbable polymers, copolymers or polymer mixtures or alloys. Preferably, tissue arrows 20 and 30 are fabricated from a bioabsorbable polymer, 80L/20 D,L PLA, which is a copolymer of L-lactide and D-lactide. Preferably, flexible connecting member 40 is a braided or mono-filament suture. The suture material can be fabricated of any suitable material that is either permanent or bioabsorbable. For example, flexible connecting member 40 could formed of an elastomeric material. Preferably, flexible connecting member 40 is fabricated of a non-absorbable polyester, size # 0.

Suture arrow device 10 may be manufactured with melt molding methods known in the prior art. It is also possible to use the techniques of U.S. Pat. No. 4,743,257, hereby incorporated by reference, to mold in a compression or injection mold absorbable fibers and binding polymer together to create a fiber-reinforced or especially a self-reinforced structure. Suture arrow device 10 may be molded in a single compression molding cycle, or the protrusions of second tissue arrow 30 may be machined on the surface of a device after the molding cycle.

The oriented and/or self-reinforced structure may also be created during extrusion or injection molding of absorbable polymeric melt trough a suitable die or into a suitable mold at high speed and pressure. When cooling occurs at suitable conditions, the flow orientation of the melt remains in the solid material as an oriented or self-reinforcing structure. In an embodiment, the mold may have the form of suture arrow device 10, but it is also possible to manufacture the suture arrow device 10 by machining (possibly using heat) and thermoforming (e.g. by bending the proximal end) of injection-molded or extruded semi-finished products.

Preferably, suture arrow device 10 is made of melt-molded, solid-state drawn or compressed, bioabsorbable polymeric materials, which are described e.g. in U.S. Pat. No. 4,968,317 or 4,898,186, both hereby incorporated by reference.

The reinforcing fibers used to make a fiber-reinforced suture arrow device 10 may also be ceramic fibers, like bioabsorbable hydroxyapatite or bioactive glass or tricalcium phosphate fibers. Such bioabsorbable, ceramic fiber reinforced materials are described e.g. in European Patent Application No. 0146398 and in WO 96/21628.

An oriented and/or self-reinforced or otherwise fiber reinforced suture arrow device 10 of this invention may be manufactured by molding the reinforcement fiber-polymer matrix to the final product in a mold, whose mold cavity has the form of the final product or the final form may be machined mechanically (possibly also using heat) on a preform, such as a melt-molded and solid-state drawn rod, as is described e.g. in U.S. Pat. No. 4,968,317.

The reinforcement elements may extend into any protrusions or ridges of second tissue arrow 30. The reinforcement elements may also turn spirally around the long axis of the implantation members and/or of the connecting member. Also other different orientations of reinforcement elements in elongated samples, which are familiar from composite technology, may be applied to the present invention. However, a general feature of orientation and/or fiber-reinforcement or self-reinforcement of a suture arrow device 10 of the present invention is that many of the reinforcing elements are oriented in such a way that they may carry effectively the different external loads (such as tensile, bending and shear loads) that are directed to the healing rupture (for example loads to a meniscus caused by the movements of the patient's knee).

Oriented and/or reinforced materials that may be used to manufacture a suture arrow device 10 of the present invention typically have initial tensile strengths of 100-2000 MPa, bending strengths of 100-600 MPa and shear strengths of 80-400 MPa. Additionally, they may be made stiff and tough or flexible.

According to an embodiment of the invention, suture arrow device 10, or a special coating layer on its surface, may contain one or more bioactive substances, such as antibiotics, chemotherapeutic substances, angiogenic growth factors, substances accelerating the healing of the wound, growth hormones and the like.

Suture arrow device 10 of the present invention may be sterilized by any of the well known sterilization techniques, depending on the type of material used in manufacture of the device. Suitable sterilization techniques include heat or steam sterilization, radiation sterilization such as cobalt 60 irradiation or electron beams, ethylene oxide sterilization, and the like.

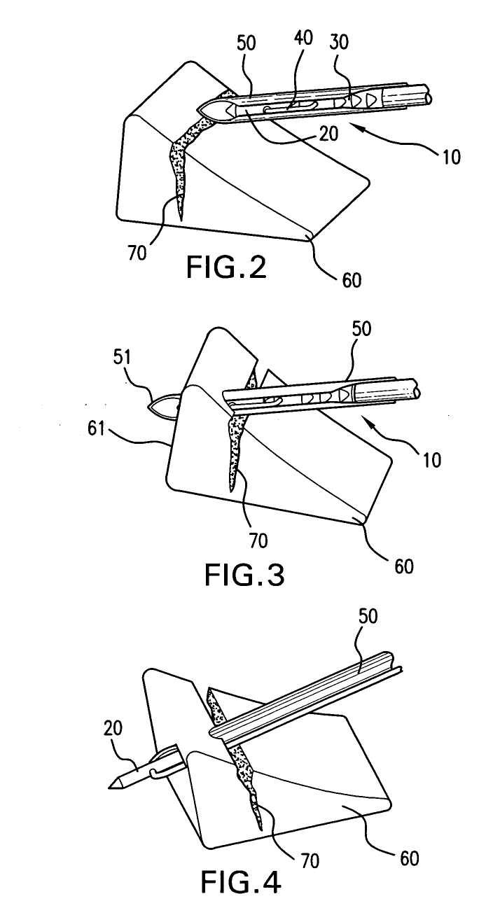

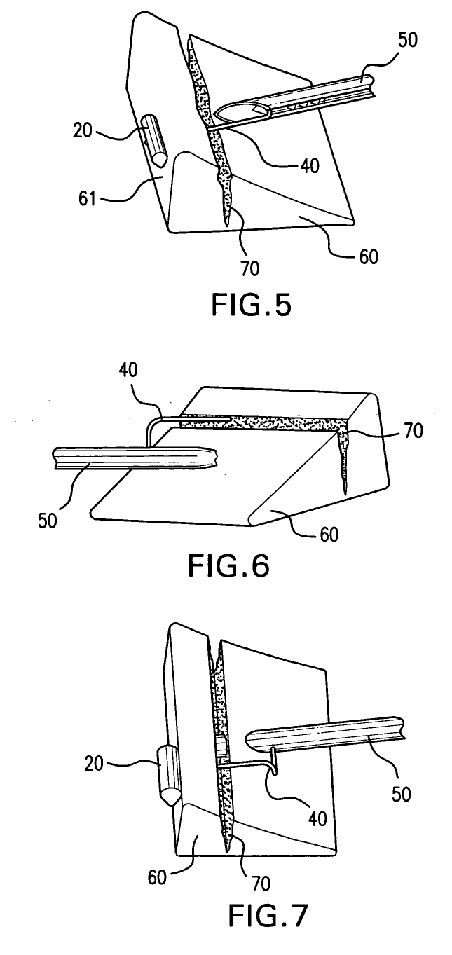

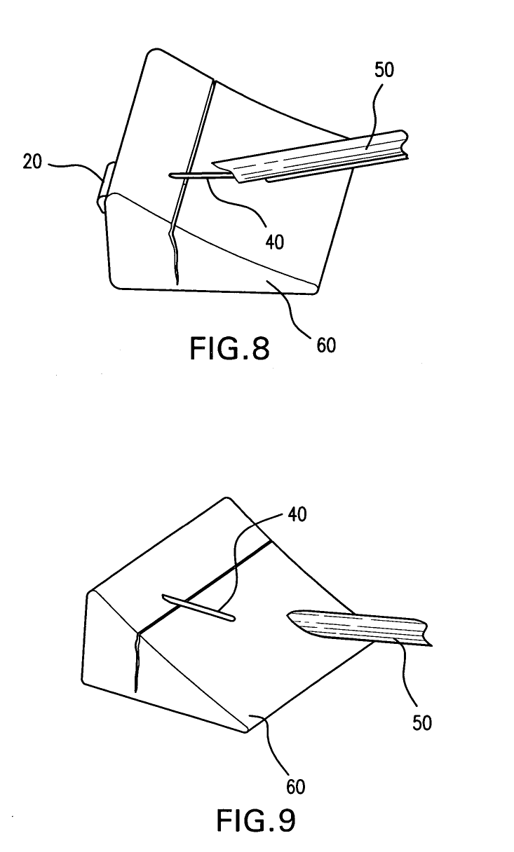

Although suture arrow device 10 is not limited to any particular method of use or installation, FIGS. 2-9 schematically illustrate an exemplary method of inserting suture arrow device 10 of the present invention in a meniscus 60 to repair a tear 70 using an exemplary installation tool 50. Referring to FIG. 2, suture arrow device 10 is first loaded into installation tool 50. Referring to FIG. 3, after loading, tip 51 of installation tool 50 is pushed through the upper part of meniscus 60 to protrude from an outer surface 61 of meniscus 60. Installation tool 50 does not necessarily need to traverse tear 70 in meniscus 60. Referring to FIG. 4, first arrow 20 is then released from tip 51 of installation tool 50 such as, for example, by pushing first arrow 20 out with a pusher rod (not shown) housed within installation tool 50. Referring to FIG. 5, first tissue arrow 20 is seated against outer surface 61 of meniscus 60 by pulling out installation tool 50 from meniscus 50, tensioning flexible connecting member 40 to rotate first tissue arrow 20 such that first tissue arrow 20 seats flush on outer surface 61. Following rotation, first tissue arrow 20 is substantially perpendicular to flexible connecting member 40. Installation tool 50 is then moved to a desirable offset location from the position where first tissue arrow 20 is seated. Referring to FIG. 6, installation tool 50 is then pushed into meniscus 60 at the selected offset location. Referring to FIG. 7, installation tool is pushed across tear 70 but not entirely through meniscus 60 as with the insertion of first tissue arrow 20. Referring to FIG. 8, second tissue arrow 30 (not shown) is inserted either across tear 70 or at another desirable portion of meniscus 60 and second tissue arrow 30 is deployed. Preferably, at least one of first tissue arrow 20 and second tissue arrow 30, and more preferably second tissue arrow 30, traverses tear 70 on insertion. Referring to FIG. 9, after second tissue arrow 30 has been deployed and tear 70 has been closed, installation tool 50 is then pulled back and removed from meniscus 60 (full removal of installation tool from meniscus 60 is not shown).

The insertion of second tissue arrow 30 at an appropriate point that is far enough from first tissue arrow 20 and deep enough in meniscus 60 results in a taut flexible connecting member 40 connecting first and second tissue arrows 20 and 30 and results in closure of tear 70. If flexible connecting member 40 is not sufficiently taut after insertion of second tissue arrow 30, a separate pusher rod (or equivalent structure), for example, can be employed to insert second tissue arrow 30 deeper into meniscus 60. Once tear 70 is satisfactorily closed and flexible connecting member 40 is sufficiently taut, any extra flexible extending proximally from second tissue arrow 30 (and indicated as 40a in FIG. 1) is cut away. Since first arrow 20 seats on outer surface 61 of meniscus 60, such a position prevents pull out of first arrow 20. Further, second arrow 30 is not deployed in the hole created for it, but rather is pushed through meniscus 60 after being brought within it by installation tool and second arrow 30 relies on protrusions 50 to prevent pull out from meniscus 60.

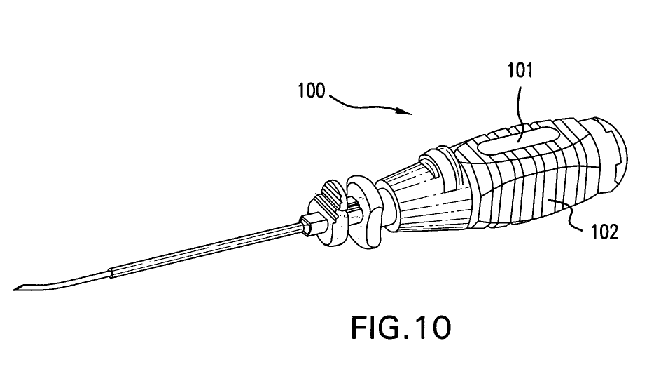

Other embodiments of the present invention provide an installation device for inserting suture arrows, such as a suture arrow device comprising a first and second arrow connected by a suture (i.e. an “H” shaped fastener), into the body. Referring to FIG. 10, which illustrates an embodiment of an installation device 100 according to the present invention in a non-operative position, the terms “lateral surface” or “side surface,” and “top surface” as used herein, refer to surfaces of installation device 100 or components of installation device 100 (described in more detail below) when installation device 100 is in a resting position. Specifically, “lateral surface” or “side surface” of installation device 100 is indicated by numeral 102 (installation device 100 also has an opposing lateral or side surface 102 not shown) and “top surface” is indicated by numeral 101.

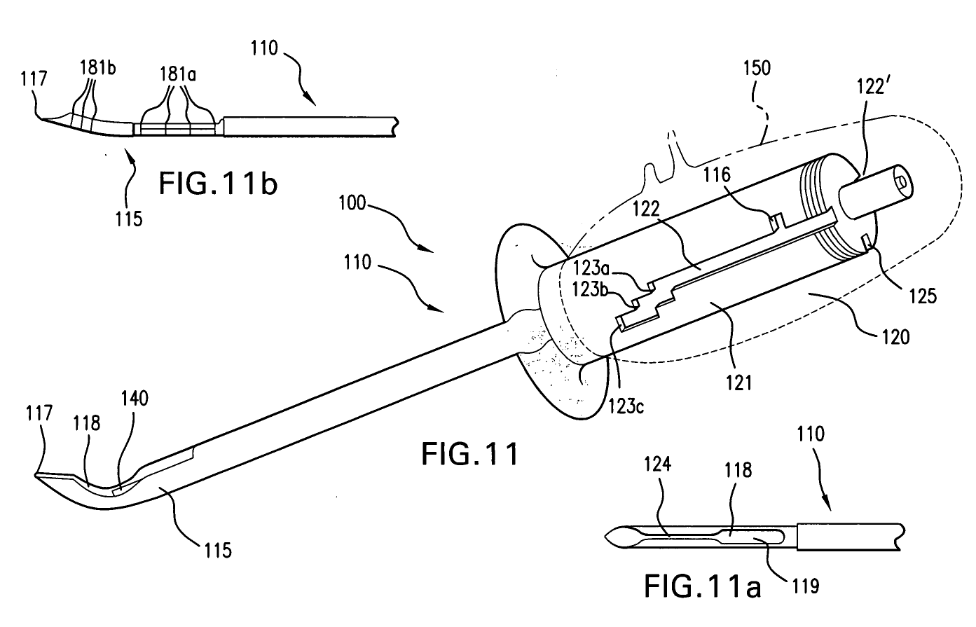

One embodiment of installation device 100 of the present invention is collectively illustrated in FIGS. 11-14. Referring to FIG. 11, installation device 100 comprises an elongated cannulated slide piece 110 having a distal portion 115 and a proximal portion 120. Distal portion 115 terminates in a sharp tip 117 and preferably distal portion 115 defines a longitudinally extending slot 118. Referring to FIG. 11a, even more preferably, longitudinally extending slot 118 has a proximal section 119 and a distal section 124 that is narrower than proximal section 119. More preferably, distal portion 115 of slide piece 110 is a removable needle. Referring to FIG. 11b, preferably, distal portion 115 of slide piece 110 has depth markings 181 etched thereon to facilitate placement of suture arrows. Such depth markings mark the distance from tip 117 to the respective depth marking. For example, if installation device 100 is used to insert a suture arrow device comprising a first and second arrow, distal portion 115 of slide piece 110 may have two sets of depth markings, 181a and 181b. Proximal set 181a is used to facilitate placement of the first arrow and distal set 181b is used to facilitate placement of the second arrow. Although there can be any number of depth markings 181, which can indicate any distance from tip 117, preferably, set 181a has four depth markings indicating a distance of 19 millimeters (mm), 24 mm, 29 mm, and 34 mm from tip 117 and set 181b has three depth markings indicating a distance of 6 mm, 8 mm, and 10 mm from tip 117.

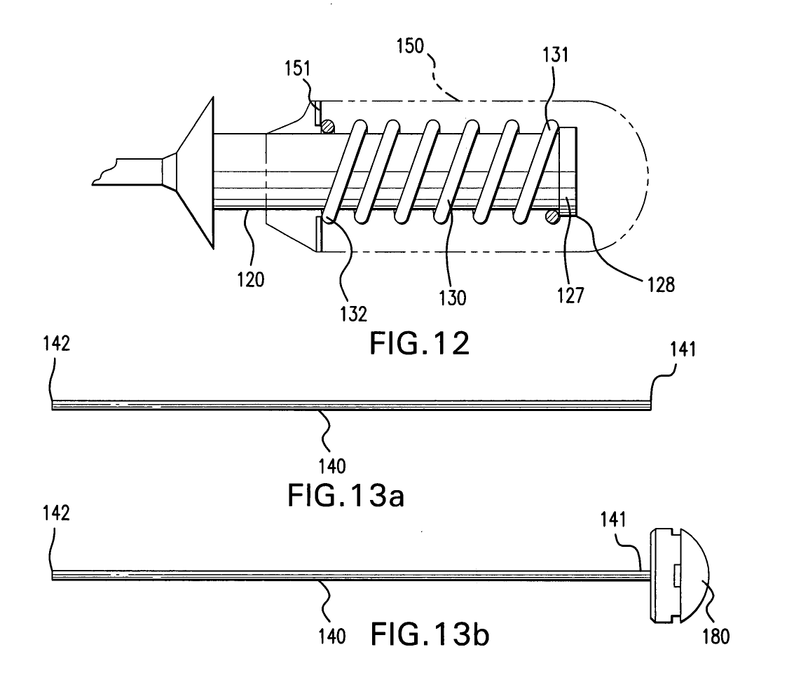

Referring to FIGS. 11 and 12, proximal portion 120 of slide piece 110 has a lateral surface 121 defining a longitudinally extending groove 122. Groove 122 has sequential gradations 123 and a notch 116 proximal of sequential gradations 123. Proximal portion 120 may also have an opposing lateral surface that is the mirror image of lateral surface 121. Specifically, such an opposing lateral surface also defines a longitudinal extending groove 122′ having sequential gradations and a notch proximal of the sequential gradations. As illustrated in FIG. 12, a spring 130 is located about proximal portion 120. Preferably, slide piece 110 includes a cap 127 at its proximal end 128 and spring 130 has a first end 131 abutting cap 127.

Referring to FIGS. 11 and 13, installation device 100 further comprises an elongated pusher rod 140 which is housed within needle member 110. Pusher rod 140 has a proximal end 141 and a distal end 142.

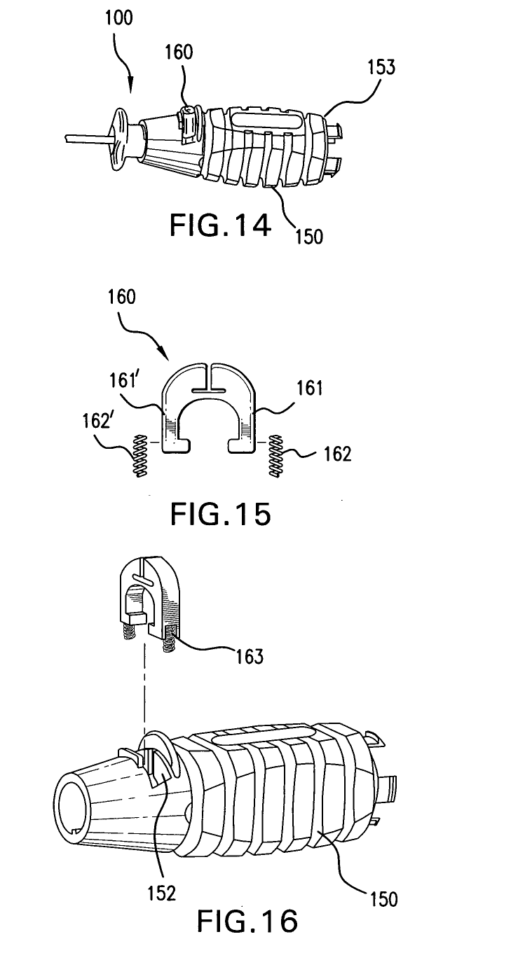

Referring to FIGS. 11, 12, 14, and 16 installation device 100 further comprises a casing 150, which is depicted in phantom lines in FIGS. 11 and 12, disposed about proximal portion 120 of slide piece 110. Referring to FIG. 12, preferably casing 150 has an inner rim 151 and spring 130 has a second end 132 abutting inner rim 151 such that spring 130 is positioned between cap 127 at proximal end 128 of slide piece 110 and inner rim 151 of casing 150. A button 160, illustrated in FIG. 14, is housed by housing 152 preferably defined by a top surface of casing 150, as illustrated in FIG. 16. Button 160 is assessable to groove 122 of proximal portion 120 of slide piece 110. Referring to FIG. 15, button 160 has an arm 161, as well as an opposing arm 161′ in embodiments of installation device 100 having an opposing lateral surface. In such embodiments, arms 161 and 161′ are the parts of button 160 that are accessible to groove 122 and groove 122′, respectively, of proximal portion 120 of slide piece 110. Referring to FIG. 16, arms 161 and 161′ also define grooves 163 and 163′ (not shown) for receiving springs 162 and 162′.

Referring to FIGS. 13b and 14, preferably installation device 100 further comprises an end cap 180 that is attachable to proximal end 141 of pusher rod 140 and back end 153 of casing 150.

Preferably, proximal portion 120 of slide piece 110 and casing 150 have mechanisms by which they can be aligned with each other. For example, referring back to FIG. 11, to assist in aligning casing 150 about proximal portion 120 of slide piece 110, casing 150 may have an alignment rail and proximal portion 120 of slide piece 110 may have a longitudinally extending channel 125 configured to ride the alignment rail of casing 150. Alternatively, proximal portion 120 of slide piece 110 may have the alignment rail and casing 150 may have the longitudinally extending channel to align proximal portion 120 of slide piece 110 and casing 150.

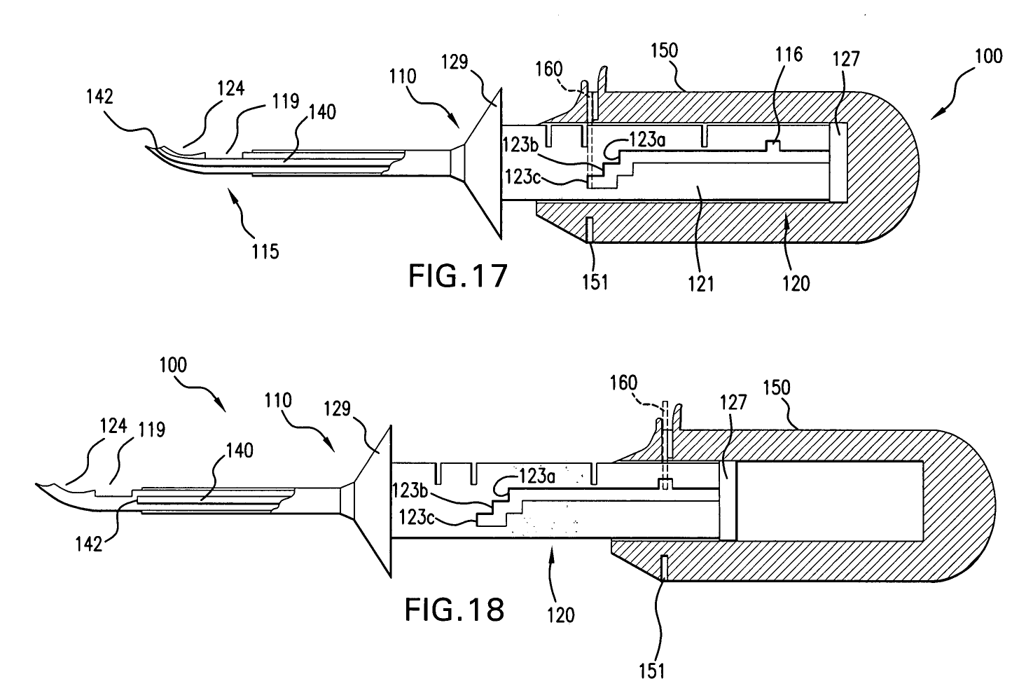

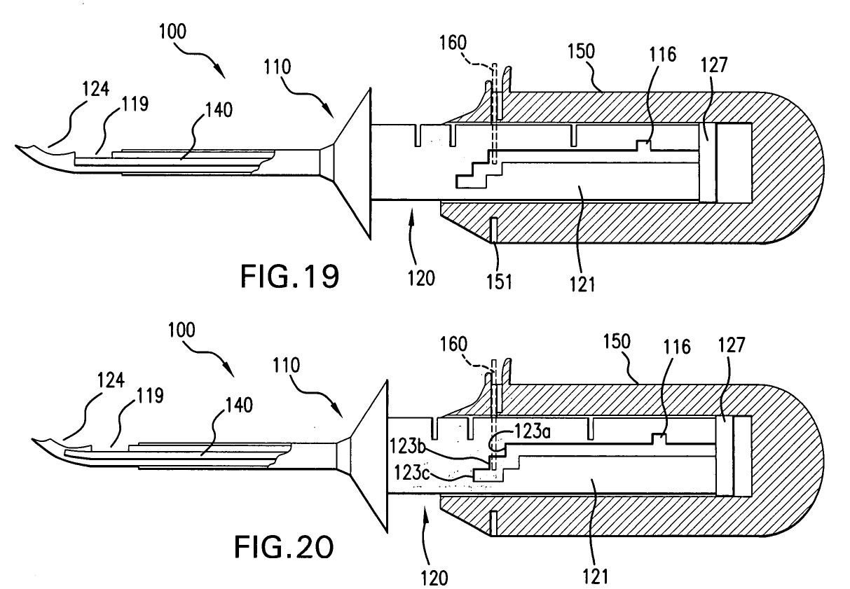

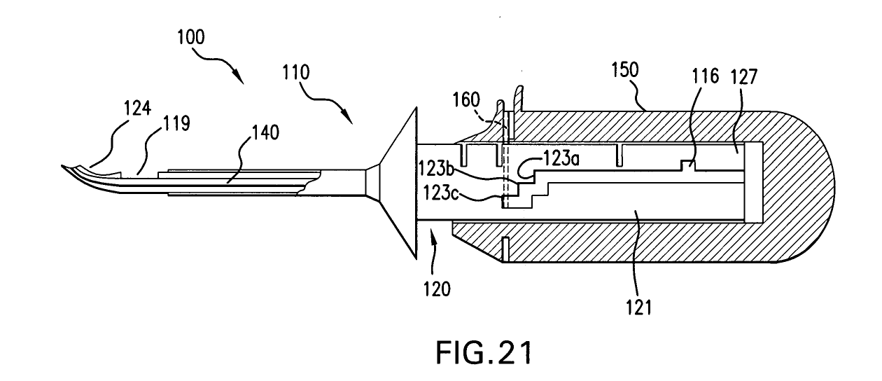

An exemplary method of using this embodiment of installation device 100 is illustrated in FIGS. 17-21. For purposes of this description, proximal section 119 of slot 118 will be referred to as the “loading slot” and distal section 124 of slot 118 will be referred to as the “deployment slot.” Referring to FIG. 17, in a non-operative resting position, arm 161 (not shown) of button 160 (depicted in broken lines for purposes of clarity) on casing 150 is at one of gradations 123 and the distal end 142 of pusher rod 140 is positioned in the deployment slot 124 of slot 118 such that loading slot 119 is in a “closed” position. In FIG. 17, arm 161 is depicted at gradation 123c such that spring 130, which abuts cap 127 at proximal end 128 of slide piece 110 and inner rim 151 of casing 150, is in its resting, unbiased position. To bring loading slot 119 in an “open” position, the user slides slide piece 110 in a distal direction until arm 161 of button 160 springs into notch 116 of proximal portion 120 of slide piece 110, as illustrated in FIG. 18. To facilitate the ability of the user to slide out proximal portion 120 of slide piece 110 from casing 150, slide piece 110 may have a shoulder 129, which the user can grasp while sliding out proximal portion 120. As depicted in FIG. 18, distal end 142 of pusher rod 140 is now positioned proximal of loading slot 119 such that loading slot 119 is in an open position. At this stage, spring 130 is outwardly biased to its resting position. A suture arrow device comprising a first and second arrow is now loaded into loading slot 119 with the second arrow lined up behind the first arrow. Button 160 is then pressed and arm 161 is released from notch 116 allowing spring 130 to move towards its resting position bringing slide piece 110 further proximally into casing 150, as illustrated in FIG. 19. Arm 161 of button 160 is now positioned at gradation 123a. Because slide piece 110 has been proximally shifted in relation to pusher rod 140, the second arrow pushes against pusher rod 140, moving first and second arrows into deployment slot 124. Loading slot 119 is now in a closed position. Referring to FIG. 20, to release the first arrow into the desired target site of the tissue to be repaired, button 160 is pressed allowing spring 130 to move further towards its resting position bringing elongated needle member further proximally into casing 150 until gradation 123b reaches arm 161 of button 160. Because slide piece 110 has been proximally shifted in relation to pusher rod 140, the second arrow pushes against distal end 142 of pusher rod 140, which causes the second arrow to move forward and apply force to the first arrow, expelling the first arrow from deployment slot 124 into the target site. Installation device 100 is then removed from the target site and re-positioned to another target site of the tissue for deployment of the second arrow. Referring to FIG. 21, to release the second arrow into the another target site, button 160 is pressed allowing the spring to move to its resting position bringing elongated needle member further proximally into casing 150 until gradation 123c reaches arm 161 of button 160. Because slide piece 110 has been proximally shifted in relation to pusher rod 140, the second arrow pushes against distal end 142 of pusher rod 140, expelling the second arrow from deployment slot 124 into the another target site.

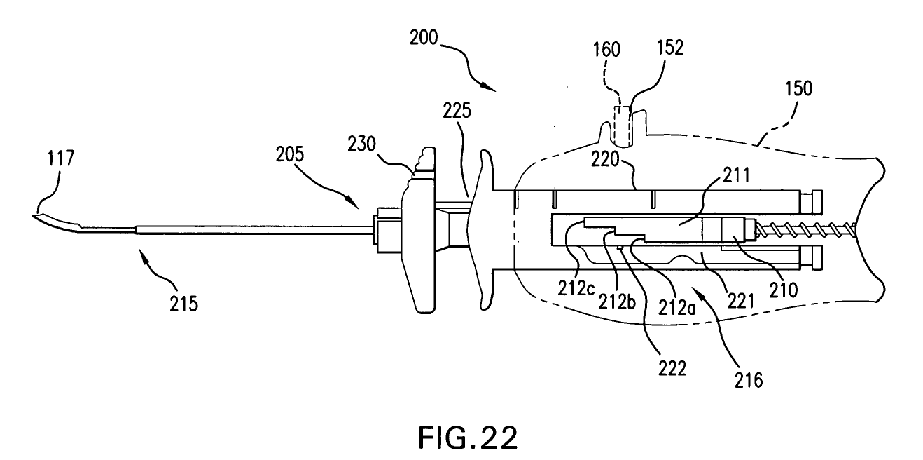

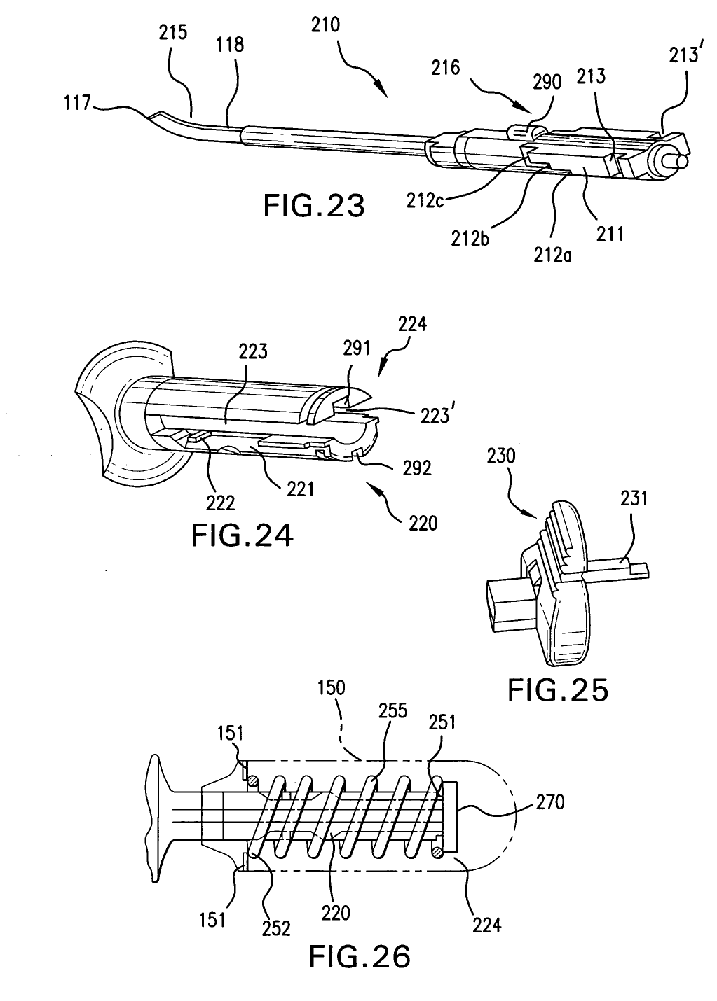

Another embodiment of an installation device is illustrated in FIGS. 22-28. Referring to FIG. 22, in this embodiment, installation device 100 comprises a slide assembly 205 comprising an elongated cannulated inner slide piece 210 and an outer slide piece 220. Referring to FIG. 23, inner slide piece 210 has a distal portion 215 and a proximal portion 216. Distal portion 215 terminates in a sharp tip 117 and preferably distal portion 215 defines a longitudinally extending slot 118 (depicted in FIG. 23). As with the embodiment of installation device 100 described above and illustrated in FIG. 11a, even more preferably, longitudinally extending slot 118 has a proximal section 119 and a distal section 124 that is narrower than proximal section 119. Further, as with the embodiment of installation device 100 described above and illustrated in FIG. 11b, preferably distal portion 215 of slide piece 210 has depth markings 181 etched thereon to facilitate placement of suture arrows. Proximal portion 216 of inner slide piece 210 has a lateral surface 211 defining sequential gradations 212 and a notch 213 proximal of sequential gradations 212. Proximal portion 216 of inner slide piece 210 may also have an opposing lateral surface that is the mirror image of lateral surface 211. Specifically, such an opposing lateral surface also defines sequential gradations and a notch 213′ proximal of the sequential gradations.

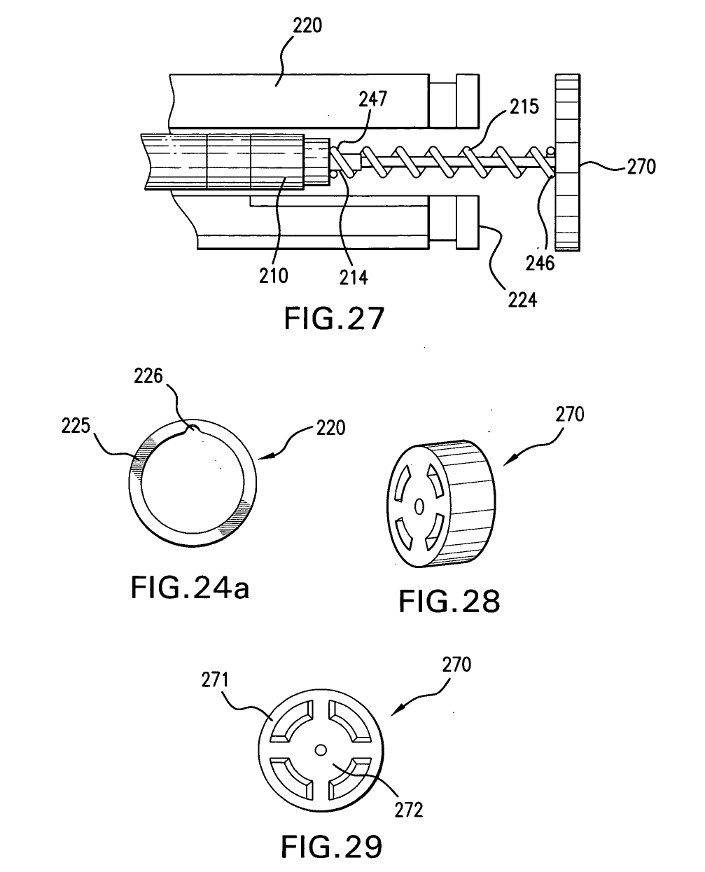

Referring to FIG. 22 again, slide assembly 205 further comprising an outer slide piece 220 disposed about proximal portion 216 of inner slide piece 210. Referring to FIG. 24, outer slide piece 220 has a side surface 221 that includes a flange 222 and that defines a longitudinally extending slit 223. As seen in FIG. 22, longitudinally extending slit 223 exposes lateral surface 211 of proximal portion 216 of inner slide piece 210. Outer slide piece 220 may also have an opposing side surface that is the mirror image of side surface 221. Specifically, such an opposing side surface also includes a flange and defines a longitudinally extending slit 223′. Referring to FIG. 24a, preferably outer slide piece 220 defines a keyway 226 with an entrance at distal end 225 of outer slide piece 220.

Referring to FIG. 13, installation device 100 further comprises an elongated pusher rod 140, which is housed within inner slide piece 210 and has a proximal end 141 and a distal end 142.

Referring to FIG. 22, installation device 100 further comprises a casing 150, which is depicted in phantom lines in FIG. 22 disposed about outer slide piece 220. As illustrated in FIG. 26, preferably casing 150 has an inner rim 151 (described below). A button 160 is housed by housing 152 defined preferably by a top surface of casing 150 and is similar in structure to button 160 described in the previous embodiment of installation device 100 described above and illustrated in FIG. 15. Button 160 of the present embodiment of installation device 100 is assessable to lateral surface 211 of proximal portion 216 of inner slide piece 210 and side surface 221 of outer slide piece 220.

Referring to FIG. 22, installation device 100 further comprises a tab member 230 located about inner slide piece 210, preferably distal of lateral surface 211 of proximal portion 216. Referring to FIG. 25, tab member 230 preferably comprises a stopper 231 that is contactable with distal end 225 and receivable by keyway 226 of outer slide piece 220.

As illustrated in FIG. 26, installation device 100 of this embodiment further comprises an outer spring 255 that is located about outer slide piece 220 and, as illustrated in FIG. 27, an inner spring 245 at a distal end 214 of inner slide piece 210. In order to secure both outer spring 255 and inner spring 245 within installation device 100, preferably installation device 100 further comprises a cap 270 (shown in isolation in FIGS. 28 and 29) securable to a proximal end 224 of outer slide piece 220. Referring to FIG. 29, cap 270 has an outer rim 271 and a coaxial inner rim 272. A proximal end 251 (depicted in FIG. 26) of outer spring 255 abuts outer rim 271 of cap 270 and a distal end 252 of outer spring 255 abuts inner rim 151 of casing 150. With respect to inner spring 245, a proximal end 246 (depicted in FIG. 27) of inner spring 245 abuts inner coaxial rim 272 of cap 270 and a distal end 247 of inner spring 245 is in contact with a proximal end 214 of inner slide piece 210. Preferably, proximal end 214 of inner slide piece 210 is in the shape of a peg such that distal end 247 of inner spring 245 can be disposed about proximal end 214 as illustrated in FIG. 27.

Preferably, installation device 100 further comprises an end cap 180, similar to the previous embodiment of installation device 100 and depicted in FIG. 13b that is attachable to proximal end 141 of pusher rod 140 (as illustrated in FIG. 13b) and back end 153 of casing 150.

Preferably, the components of installation device 100 that are in communication with each other have mechanisms by which a component can be aligned with the respective other components. For example, referring back to FIGS. 23 and 24, to assist in aligning outer slide piece 220 about inner slide piece 210, preferably inner slide piece 210 has an alignment rail 290 and outer slide piece 220 defines a longitudinally extending channel 291 configured to ride alignment rail 290 of inner slide piece 210. Similarly, to assist in aligning casing 150 about outer slide piece 220, preferably casing 150 has an alignment rail and outer slide piece 220 defines a longitudinally extending channel 292 configured to ride the alignment rail of casing 150. Of course, in any of these embodiments, the component having the alignment rail can instead define the longitudinally extending channel and the component defining the longitudinally extending channel can have the alignment rail.

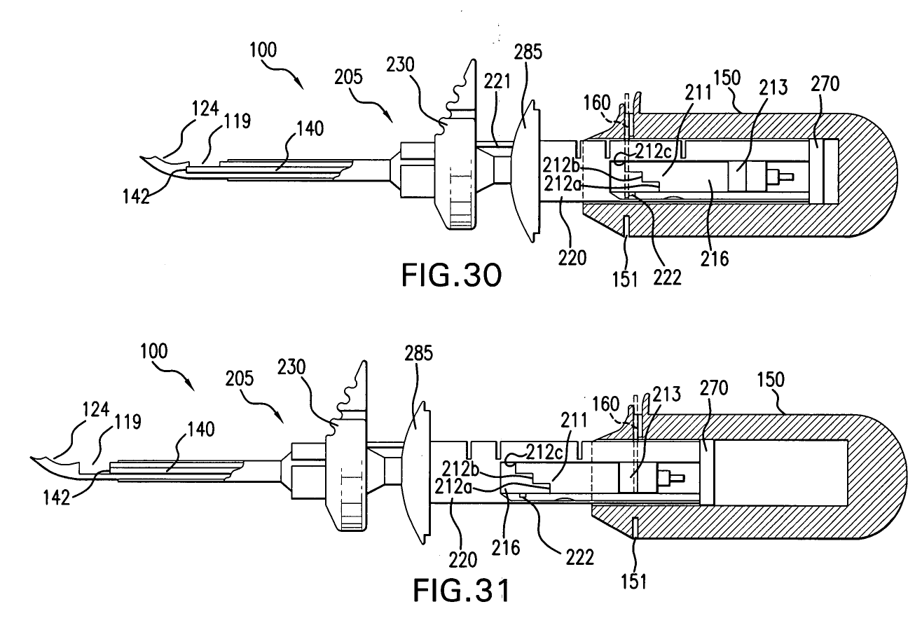

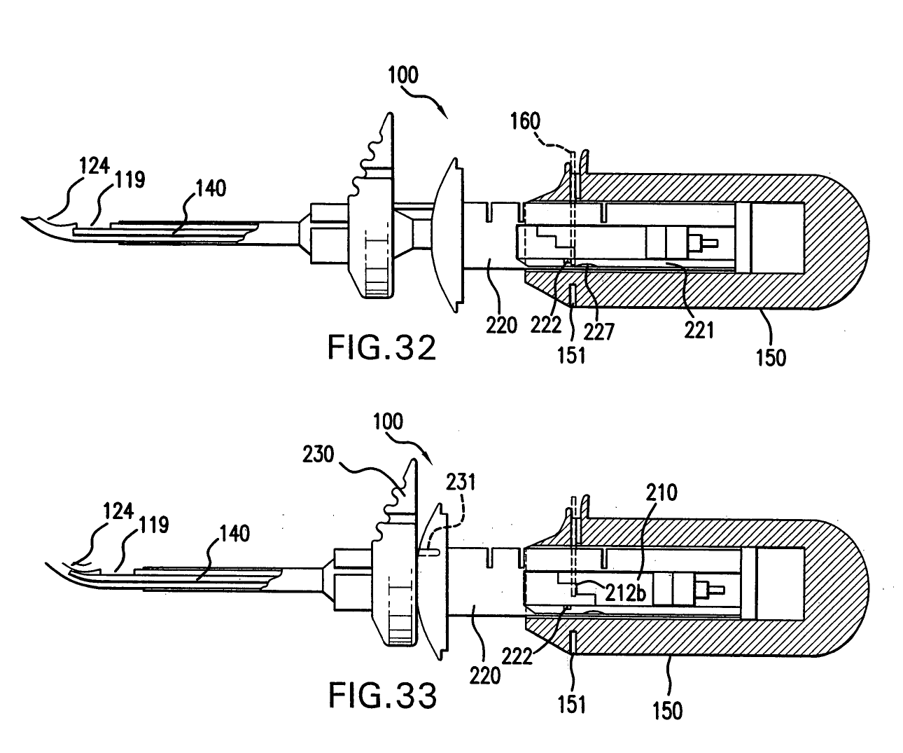

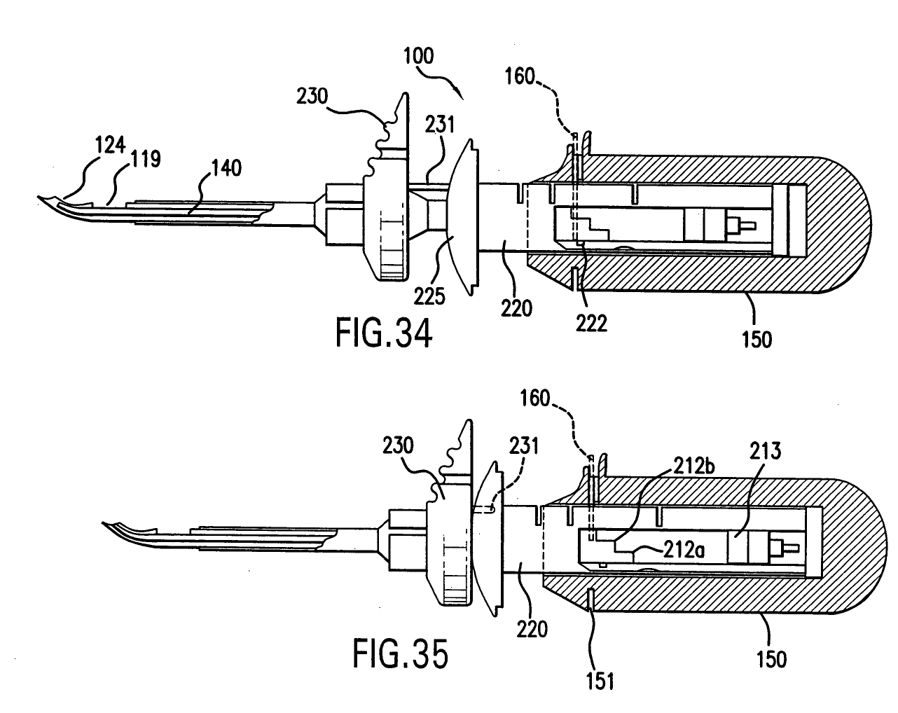

An exemplary method of using this embodiment of installation device 100 is illustrated in FIGS. 30-36. For purposes of this description, proximal section 119 of slot 118 will be referred to as the “loading slot” and distal section 124 of slot 118 will be referred to as the “deployment slot.” Referring to FIG. 30, in a non-operative position, button 160 (shown in broken lines for purposes of clarity) on casing 150 rests against a distal side of flange 222 of outer slide piece 220 and the distal end 142 of pusher rod 140 is positioned at approximately the midpoint of deployment slot 124 of slot 118 such that loading slot 119 is in a “closed” position. Outer spring 255 (not shown), which abuts inner rim 151 of casing 150 and outer rim 271 of cap 270, is in an un-biased resting position. Inner spring 245 (also not shown), which abuts proximal end 214 of inner slide piece 210 and inner rim 272 of cap 270, is also in an un-biased resting position. To bring loading slot 119 in an “open” position, button 160 is pressed and the user slides slide assembly 205 in a distal direction until arm 161 (not shown) of button 160 springs into notch 213 of proximal portion 216 of inner slide piece 210, as illustrated in FIG. 31. To facilitate the ability of the user to slide out slide assembly 205, outer slide piece 220 may have a shoulder 285, which the user can grasp while sliding out slide assembly 205. As depicted in FIG. 31, distal end 142 of pusher rod 140 is now positioned proximal of loading slot 119 such that loading slot 119 is in an open position. At this stage, outer spring 255 is outwardly biased to its resting position. A suture arrow device comprising a first and second arrow is now loaded into loading slot 119 with the second arrow lined up behind the first arrow. Button 160 is then pressed and arm 161 is released from notch 213 allowing outer spring 255 to move towards its resting position bringing slide assembly 205 further proximally into casing 150, as illustrated in FIG. 32. Arm 161 of button 160 is now positioned against a proximal side of flange 222. As depicted in FIG. 32, in order to position arm 161 against proximal side of flange 222, side surface 221 of outer slide piece 220 preferably defines a rail 227 that arm 161 contacts to direct arm 161 to the proximal side of flange 222. At this stage, distal end 142 of pusher rod 140 is at approximately the distal most end of loading slot 119, as illustrated in FIG. 32 and the first and second arrows have now been pushed into deployment slot 124 and loading slot 119 is in a closed position. Referring to FIG. 33, to release the first arrow into the desired target site of the damaged tissue, tab member 230 is pulled back (stopper 231 enters keyway 226 of outer slide piece 220) retracting inner slide piece 210, which causes arm 161 of button 160 to spring to gradation 212b (on the proximal side of flange 222). At this stage, inner spring 245 is inwardly biased to its resting position. Because inner slide piece 210 has been proximally shifted in relation to pusher rod 140, the second arrow pushes against distal end 142 of pusher rod 140, which causes the second arrow to move forward and apply force to the first arrow, expelling the first arrow from deployment slot 124 to the target site. Installation device 100 is then re-positioned to another target site of the damaged tissue for deployment of the second arrow. Button 160 is pressed releasing arm 161 from gradation 212b allowing inner spring to move towards its resting position which pushes the inner slide piece 210 in a distal direction thereby bringing stopper 231 back against distal end 225 of outer slide piece 220 as depicted in FIG. 34. Arm 161 moves under flange 222 allowing the outer spring to move to its resting position brining slide assembly 205 further proximally into casing 150 and arm 161 springs into gradation 212b (on the distal side of flange 222) as depicted in FIG. 34. To deploy the second arrow, tab member 230 is pulled back (stopper 231 enters keyway 226 of outer slide piece 220) retracting inner slide piece 210, which causes arm 161 of button 160 to spring to gradation 212c (on the proximal side of flange 222). Because inner slide piece 210 has been proximally shifted in relation to pusher rod 140, the second arrow pushes against distal end 142 pusher rod 140, which causes the second arrow to be expelled from deployment slot 124 to the another target site.

The foregoing description and examples have been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

1. An installation device comprising:
an elongated cannulated slide piece having a distal portion and a proximal portion, the distal portion terminating in a sharp tip and the proximal portion having a lateral surface defining a longitudinally extending groove, the groove having sequential gradations and a notch proximal of the sequential gradations;
a spring located about the proximal portion of the slide piece;
a pusher rod housed within the slide piece;
a casing disposed about the proximal portion of the slide piece; and
a button housed by the casing that is assessable to the longitudinally extending groove of the lateral surface of the proximal portion of the slide piece.

2. The installation device of claim 1, wherein the proximal portion of the slide piece further comprises an opposing lateral surface defining a longitudinally extending groove, the groove having sequential gradations and a notch proximal of the sequential gradations.

3. The installation device of claim 2, wherein the button has an arm that is accessible to the longitudinally extending groove of the lateral surface of the slide piece and an opposing arm that is accessible to the longitudinally extending groove of the opposing lateral surface of the slide piece.

4. The installation device of claim 1, wherein the distal portion of the slide piece defines a longitudinally extending slot.

5. The installation device of claim 4, wherein the longitudinally extending slot has a proximal section and a distal section, the distal section being narrower than the proximal section.

6. The installation device of claim 1, further comprising a cap at a proximal end of the slide piece.

7. The installation device of claim 6, wherein the casing has an inner rim and the spring has a first end abutting the cap and a second end abutting the inner rim of the casing.

8. An installation device comprising:
a slide assembly comprising:
an elongated cannulated inner slide piece having a distal portion and a proximal portion, the distal portion terminating in a sharp tip and the proximal portion having a lateral surface, the lateral surface defining sequential gradations and a notch proximal of the sequential gradations; and
an outer slide piece disposed about the proximal portion of the inner slide piece having a side surface including a flange and defining a longitudinally extending slit that exposes the lateral surface of the proximal portion of the inner slide piece;
an outer spring located about the outer slide piece;
an inner spring at a proximal end of the inner slide piece;
a tab member located about the inner slide piece;
a pusher rod housed within inner slide piece; and
a casing disposed about the outer slide piece;
a button housed by the casing that is assessable to the lateral surface of the proximal portion of the inner slide piece and the side surface of the outer slide piece.

9. The installation device of claim 8, wherein the proximal portion of the inner slide piece further comprises an opposing lateral surface defining sequential gradations and a notch proximal of the sequential gradations; and the outer slide piece further comprises an opposing side surface including a flange and defining a longitudinally extending slit that exposes the opposing lateral surface of the inner slide piece.

10. The installation device of claim 8, wherein the button comprises further an arm and opposing arm, the arm accessible to the lateral surface of the inner slide piece and the side surface of the outer slide piece, the opposing arm assessable to the opposing lateral surface of the inner slide piece and the opposing side surface of the outer slide piece.

11. The installation device of claim 8, wherein the outer slide piece defines a keyway with an entrance at a distal end of the outer slide piece.

12. The installation device of claim 11, wherein the tab member comprises a stopper contactable with the distal end of the outer slide piece.

13. The installation device of claim 8, further comprising a cap at a proximal end of the outer slide piece, the cap having an outer rim and a coaxial inner rim.

14. The installation device of claim 13, wherein the casing has an inner rim.

15. The installation device of claim 14, wherein the outer spring has a proximal end abutting the outer rim of the cap and a distal end abutting the inner rim of the casing.

16. The installation device of claim 13, wherein the inner spring has a proximal end abutting the inner coaxial rim of the cap.

17. The installation device of claim 8, wherein the distal portion of the inner slide piece member defines a longitudinally extending slot.

18. The installation device of claim 17, wherein the longitudinally extending slot has a proximal section and a distal section, the distal section of the longitudinally extending slot being narrower than the proximal section of the longitudinally extending slot.

19. A suture arrow device comprising:
a first tissue arrow comprising a single, elongated body shaped like an arrow and having a pointed end;
a second tissue arrow comprising a single, elongated body shaped like an arrow and having at least one protrusion thereon and a pointed end; and
a flexible connecting member connecting the first and second tissue arrows.

20. The suture arrow device of claim 19, wherein the flexible connecting member is a suture.

21. The suture arrow device of claim 20, wherein one end of the flexible connecting member attaches at a point near the middle of the first tissue arrow and a second end of the flexible connecting member attaches a point near the distal end of the second tissue arrow but proximal to the pointed end of the second tissue arrow.

22. A method of repairing a body tissue rupture comprising:
providing a suture arrow device comprising:
a first tissue arrow;
a second tissue arrow having at least one protrusion thereon; and
a flexible connecting member connecting the first and second tissue arrows;
inserting the first tissue arrow through the body tissue;
seating the first tissue arrow on the outer surface of the body tissue such that the first tissue arrow is substantially perpendicular to the flexible connecting member; and
inserting the second tissue arrow into the body tissue in a location off-set from the first tissue arrow thereby closing the rupture.
